(19) United States
(12) Patent Application Publication
(10) Pub. No.: US 2018/0060873 A1
(43) Pub. Date: Mar. 1, 2018

(54) ELECTRONIC ATOMIZER, ANTI-COUNTERFEITING SYSTEM AND METHOD THEREOF

(71) Applicant: NUVOTON TECHNOLOGY CORPORATION, Hsinchu CIty (TW)

(72) Inventor: Ping-Ying CHU, Hsinchu City (TW)

(21) Appl. No.: 15/483,725

(22) Filed: Apr. 10, 2017

(30) Foreign Application Priority Data
Sep. 1, 2016 (TW) 105128217

(51) Int. Cl. G06Q 30/00; A24F 47/00; G06K 19/10; G06K 7/10; H04L 29/06; A61M 11/00

(52) U.S. Cl. CPC G06Q 30/018; A24F 47/008; G06K 19/10; G06K 7/10297; A61M 2205/27; A61M 11/00; G06Q 2220/00; A61M 2205/60; A61M 2205/3584; H04L 63/061

(57) ABSTRACT

An electronic atomizer capable of detecting a counterfeit liquid container is illustrated. The electronic atomizer includes a liquid container, a power supply module and a storage box. The liquid container includes an identification code carrier and an atomizing component. The liquid container accommodates fluid to be atomized, and the power supply module provides power to the atomizing component for atomization of the fluid. The storage box has a receiving space and is provided with an identifying device configured to identify the identification code carrier of the liquid container which is placed in the receiving space. Also illustrated are an anti-counterfeit system and a method thereof.

### CROSS-REFERENCE TO RELATED APPLICATION

This application claims the benefit of Taiwan Patent Application No. 105128217, filed on Sep. 1, 2016, at the Taiwan Intellectual Property Office, the disclosure of which is incorporated herein in its entirety by reference.

### BACKGROUND OF THE INVENTION

### 1. Field of the Invention

The present disclosure relates to an electronic atomizer. More particularly, the present disclosure relates to an electronic atomizer capable of identifying whether a liquid container is a counterfeit product or a genuine product.

### 2. Description of the Related Art

Electronic atomizers for healthcare use and electronic cigarettes used to replace conventional cigarettes are both able to effectively control steam and have great potential to become more popular. This is particularly the case in the cigarette market, since the electronic cigarette harms the human body much less than conventional cigarettes, due to the different nature and chemical composition used in each, and the ability of the electronic cigarette to accurately atomize E-liquid and accurately control the composition of the E-liquid.

As the electronic cigarette has become more and more popular, a wider range of compositions of higher quality E-liquids have become commercially available. E-liquids from reputable brands have good quality control in the raw materials used and the atomized steam produced in the e-cigarette, so that adsorption by the user of known undesired deleterious chemical components when vaping the electronic cigarette is completely eliminated. However, in circulation, being sold in the market, are many counterfeit E-liquids products that do not have good quality control and which mislead the consumer by masquerading as other reputable E-liquid brands. When these counterfeit E-liquids are atomized for vaping, the steam produced often contains harmful chemical components which harm the user's body when inhaled.

### SUMMARY OF THE INVENTION

In order to solve the aforementioned problems, it is a primary objective of the present disclosure to provide an electronic atomizer, an anti-counterfeit system and a method thereof.

The embodiments of the present disclosure provide an electronic atomizer including a liquid container, a power supply module and a storage box. The liquid container is configured to contain liquid to be atomized, and comprises an identification code carrier and an atomizing component. The power supply module is configured to provide power for the atomizing component to atomize the liquid. The storage box includes a receiving space and an identifying device configured to identify the identification code carrier of the liquid container which is placed in the receiving space.

Preferably, the power supply module includes a control chip, the identifying device generates an indicative signal after identifying the identification code carrier, and after the power supply module and the liquid container have been electrically coupled with each other, the control chip according to the indicative signal determines whether the power supply module provides power to the atomizing component.

Preferably, the identifying device includes a public key corresponding to a private key of the identification code carrier, and the identifying device is configured to decrypt the identification code carrier according to the public key, in order to identify a serial number stored in the identification code carrier.

Preferably, the identification code carrier is an encryption chip which includes a fuse, and the storage box includes an electrode configured to provide current to burn the fuse.

Preferably, the storage box includes an electrode configured to provide current to damage the atomizing component.

The embodiments of the present disclosure provide an anti-counterfeiting system for an electronic atomizer. The anti-counterfeiting system includes the above-mentioned electronic atomizer, a network connection module and a server. The network connection module is electrically coupled with the electronic atomizer and configured to transmit the indicative signal. The server communicates with the network connection module through a network, is configured to receive the indicative signal including a serial number, and determines whether the serial number has been registered. If the serial number has been registered, the server transmits an already-registered signal to the identifying device of the storage box, the identifying device generates a stop signal according to the already-registered signal, and upon receipt of the stop signal, the control chip stops the power supply module from providing power to the atomizing component.

The embodiments of the present disclosure provide an anti-counterfeiting method for an electronic atomizer. The electronic atomizer includes a liquid container, a storage box and a power supply module. The anti-counterfeiting method includes steps of: identifying, by an identifying device of the storage box, an identification code carrier of the liquid container to generate an identified result, and providing an indicative signal according to the identified result; electrically connecting the power supply module to the liquid container; and determining, by a control chip of the power supply module, whether the power supply module provides power to an atomizing component of the liquid container.

Preferably, the step of identifying the identification code carrier, further includes: decrypting, by the identifying device, an identification code of the identification code carrier according to a public key; when decryption fails, determining the liquid container with the identification code to be a counterfeit product and generating the indicative signal indicative of a counterfeit product; and when decryption succeeds, obtaining a serial number of the liquid container and generating the indicative signal indicative of a genuine product.

Preferably, the anti-counterfeiting method further includes the steps of: transmitting the serial number, through a network connection module connected to the identifying device, to a server; determining, by the server, whether the serial number has been registered; if the serial number has been registered, providing an already-registered signal by the server; receiving the already-registered signal, and generating a stop signal by the identifying device; and upon receipt of the stop signal, stopping the power supply module by the control chip from providing power to the atomizing component.

The embodiments of the present disclosure further provide a liquid container adapted to an electronic atomizer which includes a liquid storage chamber, an atomizing component and an identification code carrier. The liquid storage chamber is configured to accommodate fluid to be atomized. The atomizing component is configured to atomize the fluid. The identification code carrier includes encrypted content. When decryption of the encrypted content fails, the atomizing component is burnt to disable the atomizing component and prevent atomization of the fluid.

### BRIEF DESCRIPTION OF THE DRAWINGS

The structure, operating principle and effects of the present disclosure will be described in detail by way of various embodiments which are illustrated in the accompanying drawings.

FIG. 1 is a block diagram of a first embodiment of an electronic atomizer of the present disclosure.

FIG. 2 is a block diagram of a second embodiment of the electronic atomizer of the present disclosure.

FIG. 3 is a block diagram of a third embodiment of the electronic atomizer of the present disclosure.

FIG. 4 is a block diagram of a fourth embodiment of the electronic atomizer of the present disclosure.

FIG. 5 is a first flowchart showing the steps in an operation of an anti-counterfeiting method for the electronic atomizer of the present disclosure.

FIG. 6 is a second flowchart showing the steps in an operation of the anti-counterfeiting method for the electronic atomizer of the present disclosure.

FIG. 7 is a third flowchart showing the steps in an operation of the anti-counterfeiting method for the electronic atomizer of the present disclosure.

FIG. 8 is a fourth flowchart showing the steps in an operation of the anti-counterfeiting method for the electronic atomizer of the present disclosure.

### DETAILED DESCRIPTION OF THE PREFERRED EMBODIMENTS

The following embodiments of the present invention are herein described in detail with reference to the accompanying drawings. These drawings show specific examples of the embodiments of the present invention. It is to be understood that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and in the description to refer to the same or like parts.

It is to be understood that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present invention. As used herein, the term “or” includes any and all combinations of one or more of the associated listed items.

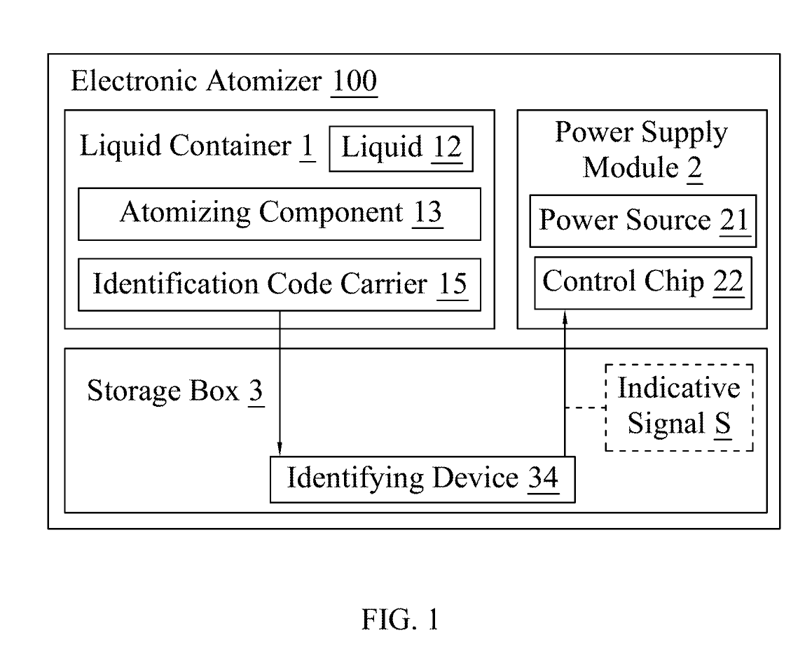

The following refers to FIG. 1, which shows a block diagram of a first embodiment of an electronic atomizer 100 of the present disclosure. The electronic atomizer 100 includes a liquid container 1, a power supply module 2 and a storage box 3. The liquid container 1 and the power supply module 2 can be placed into a receiving space of the storage box 3 via an opening of the storage box 3. The liquid container 1 is formed with a liquid storage chamber to contain liquid 12 to be atomized. Preferably, the liquid 12 can be medicine or E-liquid for an electronic cigarette; but the present disclosure is not limited thereto. Any liquid which can be atomized for use is included in the present disclosure. An atomizing component 13 is used to heat and atomize the liquid 12. It should be noted that any kind of atomization method may be implemented in the present disclosure.

For example, the atomizing component 13 may be an ultrasonicator which uses ultrasound to agitate molecules of the liquid 12, so as to implement atomization of the liquid 12. Furthermore, the atomization is not only the process of converting the liquid into micro-particles, but may also include a phase transition from the liquid to gas state. The atomized liquid can be released through an opening part of the liquid container 1.

The liquid container 1 further includes an identification code carrier 15 which serves as a device ID of the liquid container 1 for identification. For example, the identification code carrier 15 may be a serial number (SN), a barcode, an identification chip, an encryption chip or a radio frequency identification (RFID) chip; but the present disclosure is not limited thereto.

The power supply module 2 of the electronic atomizer 100 includes a power source 21, such as a battery. After the power source 21 has been electrically coupled to the atomizing component 13 of the liquid container 1, the power source 21 provides power to the atomizing component 13 for atomization of the liquid 12.

The storage box 3 includes a receiving space defined by a case which is formed with an opening, so that the liquid container 1 and/or the power supply module 2 can be placed into the receiving space of the storage box 3 through the opening of the storage box 3. An identifying device 34 can be disposed in the storage box 3 and configured to identify the identification code carrier 15 of the liquid container 1. When the liquid container 1 is placed into the receiving space of the storage box 3, the identifying device 34 can access the identification code carrier 15 of the liquid container 1 to determine whether the liquid container 1 is a genuine product or a counterfeit product. For example, when the identification code carrier 15 is the barcode, the identifying device 34 can be a barcode scanner to read content of the barcode.

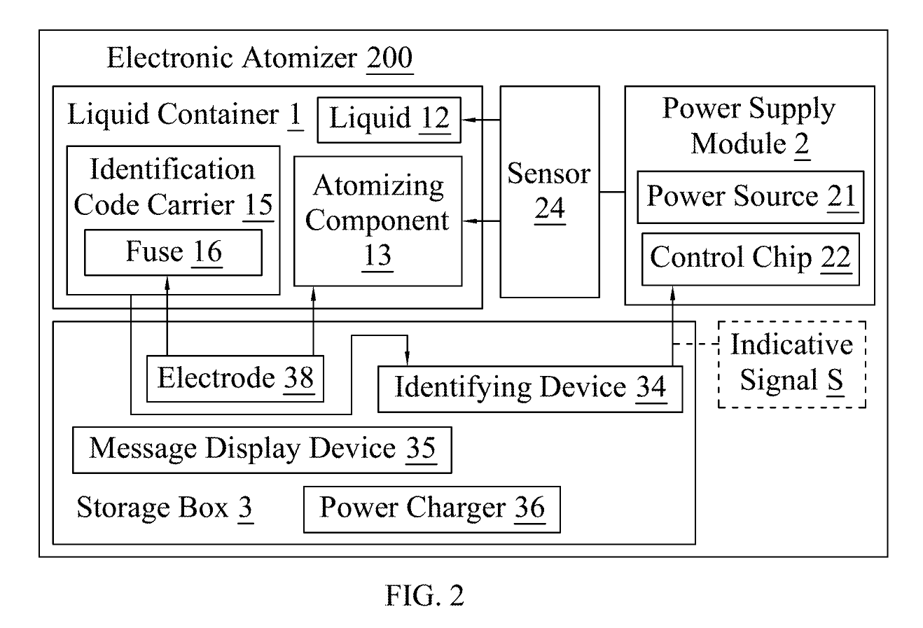

The following refers to FIG. 2, which shows a block diagram of the electronic atomizer 200 of the present disclosure. In the electronic atomizer 200, the identification code carrier 15 may be an encryption chip, and the identifying device 34 may be a decryption chip for decrypting the encryption chip of the identification code carrier 15. The encryption method applied in the present disclosure may be symmetric encryption, asymmetric encryption, digital signature, or other forms of encryption.

For example, the encryption chip may use a symmetric encryption, such as AES, DES, or 3DES, and the identification code carrier 15 may store content (such as manufacturing information or the serial number) encrypted by a cipher code. The identifying device 34 uses the same cipher code to decrypt the encrypted content, and the identifying device 34 can further verify at least one part of the decrypted content, in order to determine whether the decryption has succeeded. For example, the encrypted content may include a string of the manufacturer's name, and if the decrypted content includes the string, the decryption succeeds; otherwise, the decryption fails. However, the present disclosure is not limited to the above-mentioned manner, and any way of determining whether decryption succeeds is included in the scope of the present disclosure.

In another embodiment, the encryption manner may be an asymmetric encryption, and the identification code carrier 15 stores the content (such as manufacturing information or the serial number) which is encrypted according to a private key. The identifying device 34 uses a public key corresponding to the private key, to decrypt the encrypted content. The identifying device 34 can further verify at least one part of the decrypted content, in order to determine whether the decryption has succeeded. For example, the encrypted content may include a string of the manufacturer's name, and if the decrypted content includes the string, the decryption succeeds; otherwise, the decryption fails. Similarly, the digital signature encrypted by the public key can also be implemented in the present disclosure for encryption-decryption and identification.

The power supply module 2 further includes a control chip 22 which is configured to control various modes of the power source 21 for supplying power to the liquid container 1. For example, the control chip 22 may control the power source 21 to provide power to the liquid container 1, to not provide power to the liquid container 1, or to selectively provide power to the atomizing component 13 of the liquid container 1 according to preset conditions. When the identifying device 34 is unable to decrypt the identification code carrier 15, it indicates that the liquid container 1 is not a genuine product, so the control chip 22 of the power supply module 2 stops the power source 21 from providing power to the liquid container 1. As a result, it can prevent the user from using a non-genuine liquid container 1.

The storage box 3 can further include a message display device 35, such as at least one light-emitting diode, a screen and so on. When the identifying device 34 has decrypted the identification code carrier 15 successfully, the display device 35 shows a message indicative of completion of decryption. When the identifying device 34 is unable to decrypt, the display device 35 shows a message indicative of failure of decryption.

For example, after the consumer places the liquid container 1 into the storage box 3, the identifying device 34 starts to perform decryption on the identification code carrier 15 of the liquid container 1; if the decryption succeeds, the display device 35 may show the message indicative of completion of the decryption, and then show product information and content information of the liquid container 1. Therefore, the consumer is able to check whether the liquid container 1 is the genuine product, and then further check whether the liquid container 1 is the desired product, before the consumer atomizes the liquid stored in the liquid container 1. Otherwise, if the decryption fails, the message display device 35 may show a warning message to inform the consumer, thereby stopping the consumer from inadvertently using the counterfeit product.

Furthermore, in another embodiment, the identifying device 34 performs decryption on the identification code carrier 15 of the liquid container 1, and if decryption fails, the identification code carrier 15 can be set as inaccessible, thereby disabling usage of the liquid container 1. For example, if the identification code carrier 15 is the encryption chip which further includes a fuse 16, and when the identifying device 34 fails to decrypt the encryption chip, the fuse 16 will be burnt by the current from the electrode 38 of the storage box 3, so that the liquid container 1 cannot be used anymore and the display device 35 of the storage box 3 may then display a message to inform the user. As a result, the liquid container 1, which may be the counterfeit product, cannot then be recycled, refilled or reused.

After the decryption fails, the storage box 3 may damage the atomizing component 13 of the liquid container 1. For example, the storage box 3 provides current or voltage through the electrode 38 to damage the atomizing component 13. For example, the storage box 3 may feed current to the atomizing component 13 to burn the fuse, in order to disable the function of the atomizing component 13. Therefore, the liquid container 1, which may be the counterfeit product, cannot then be recycled, refilled, or reused.

In another embodiment, when decryption has succeeded, the user has coupled the liquid container 1 with the power supply module 2 to atomize the liquid 12, and after the liquid 12 of the liquid container 1 has been exhausted, the power supply module 2 may feed current to the atomizing component 13 to damage the atomizing component 13 or burn the fuse of the atomizing component 13. Even if the atomizing component 13 is not provided with the identification code carrier, the power supply module 2 still can feed current to damage the atomizing component 13 or burn the fuse of the atomizing component 13 after the liquid 12 is exhausted, thereby preventing the atomizing component 13 from being refilled with non-genuine liquid by a person with malicious intent.

The storage box 3 may further include a power charger 36 to charge the power supply module 2 which is placed in the receiving space of the storage box 3.

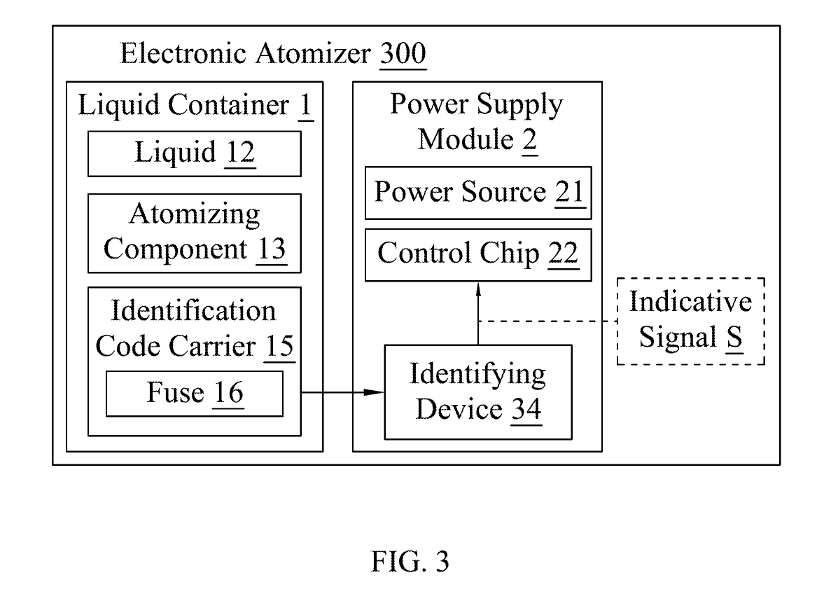

The following refers to FIG. 3, which shows a block diagram of the electronic atomizer 300 of a third embodiment of the present disclosure. The difference between the third embodiment and the first embodiment is that, in the third embodiment, the power supply module 2 includes the identifying device 34 which is configured to identify the liquid container 1. After the liquid container 1 is electrically coupled with the power supply module 2, the identifying device 34 starts to identify the identification code carrier 15. Similarly, the identification code carrier 15 may be a serial number, a barcode, an encryption chip or an RFID. The identifying device 34 may use a suitable manner to read and identify the identification code carrier 15. The ways of selecting the identifying device 34 and identifying the identification code carrier 15 are the same as that of the first embodiment, so their detailed descriptions are not repeated.

When the identifying device 34 of the power supply module 2 identifies the identification code carrier 15 of the liquid container 1 successfully, then the liquid container 1 is determined to be the genuine product. In this case, the identifying device 34 then provides the indicative signal S indicative of the genuine product to the control chip 22 of the power supply module 2, so that the control chip 22 permits the power source 21 to provide power to the atomizing component 13 for further atomization of the liquid 12 in the liquid container 1. Preferably, the control chip 22 and the identifying device 34 may be integrated in the same chip, or implemented separately by different chips.

When the identifying device 34 of the power supply module 2 is unable to identify the identification code carrier 15 of the liquid container 1, then the liquid container 1 is determined to be the counterfeit product. In this case, the identifying device 34 provides the indicative signal S indicative of the counterfeit product to the control chip 22 of the power supply module 2, so that the control chip 22 stops the power source 21 from providing power to the atomizing component 13, thereby stopping the atomization of the liquid 12 in the liquid container 1.

Similarly, when the identifying device 34 has failed to identify the identification code carrier 15, the identification code carrier 15 may be set as inaccessible, thereby stopping re-usage of the liquid container 1. For example, the power source 21 may provide current to burn the fuse 16 of the identification code carrier 15. Or, the power supply module 2 may disable the function of the atomizing component 13 of the liquid container 1 when the identification fails, and the power source 21 then provides current or voltage to damage at least one part of the atomizing component 13, in order to make the atomizing component 13 lose its function.

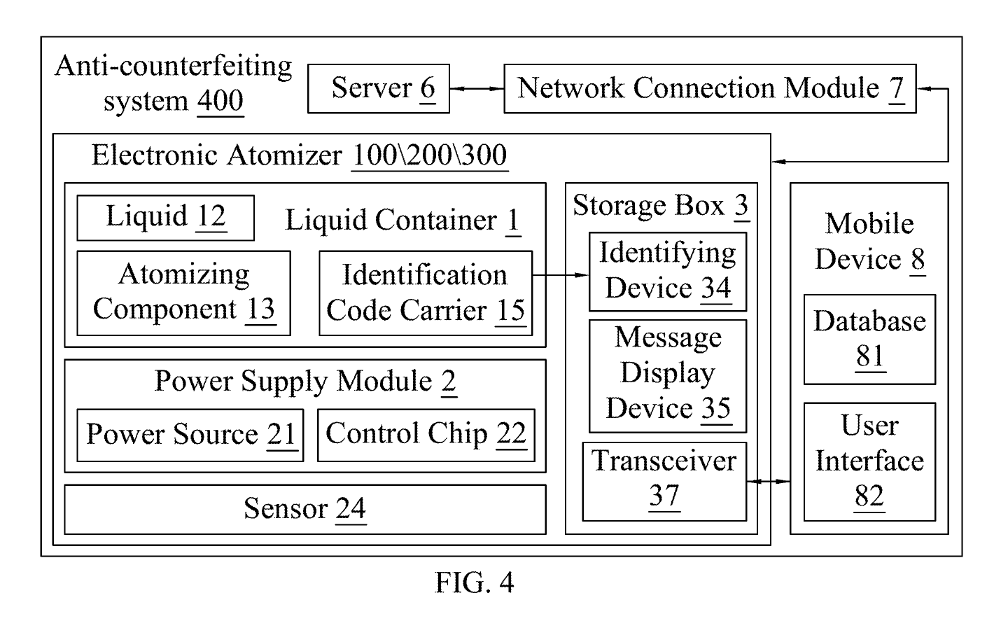

The following refers to FIG. 4, which shows an anti-counterfeiting system 400 for the electronic atomizer. As shown in FIG. 4, the anti-counterfeiting system 400 includes a server 6. The identifying device 34 attempts to identify the identification code carrier 15 of the liquid container 1, and when the identification succeeds, the storage box 3 may upload the indicative signal S (such as manufacturing information or the serial number) stored in the identification code carrier 15 to the server 6 through the interconnection between a network connection module 7 and the server 6. Preferably, the interconnection is performed by WIFI or other network technology.

After receiving the serial number, the server 6 may check whether the serial number has been uploaded and registered, in order to determine whether the liquid container 1 is the counterfeit product. If the serial number has already been registered, the server 6 provides a message to the storage box 3 through the network connection module 7, and the message will be shown on the display device 35 to inform the user that the liquid container 1 is the counterfeit product.

Like with the first embodiment, the storage box 3 may further be used to make the identification code carrier 15 inaccessible, so that the liquid container 1 cannot be reused and identified again.

Alternatively, the server 6 may transmit a stop signal to the control chip 22 of the power supply module 2 through the network connection module 7, so that the control chip 22 can stop the power supply module 2 from providing power to the atomizing component 13 after the liquid container 1 is coupled with the power supply module 2. As a result, the user is prevented from using the counterfeit liquid container 1.

If the serial number of the identification code carrier 15 has not yet been registered, the server 6 registers the serial number, and then provides information to the storage box 3 through a network, and the information may be shown on the display device 35 to inform the user that this liquid container 1 is the genuine product.

If desired, the storage box 3 may be configured to not upload the serial number to the server 6 in further instances after the first time where the identified liquid container 1 is placed into the storage box 3. For example, the serial number of the identification code carrier 15 of the liquid container 1 may be stored in the storage box 3 and the storage box 3 does not upload the content of the identification code carrier 15 to the server 6 if the identification has already succeeded once, and therefore the serial number of the liquid container 1 matches with one of the serial numbers stored in the storage box 3. Alternatively, in further instances after the first time where the content of the identification code carrier 15 of the liquid container 1 has been registered and the liquid container 1 is placed into the storage box 3, the storage box 3 does not upload the content of the identification code carrier 15 to the server 6 anymore, in order to prevent repeat registration.

In this embodiment, the anti-counterfeiting system 400 may further include a mobile device 8, and the electronic atomizer 100, 200 or 300 records the usage information of the atomizing component 13 and the liquid 12 sensed by the sensor 24, and is linked to the mobile device 8 by a wired manner, a wireless manner or through another network technology. The mobile device 8 may store the usage information in the database 81.

An electronic cigarette is taken as an example for illustration, where the liquid container 1 is an E-liquid cartridge, and the liquid 12 is an E-liquid. The sensor 24 coupled to the power supply module 2 is configured to sense the liquid 12 or the atomizing component 13 and record the usage information. For example, the usage information may include information about the remaining amount of the E-liquid calculated from the total number of sensed puffs, that is the number of sensed instances of the act of inhaling and exhaling, by which the electronic cigarette has been vaped, a location where the electronic cigarette has been vaped, the user's vaping habits including number of puffs and duration of each vaping session as well as the times and frequency of the vaping sessions. The usage information may be transmitted to the mobile device 8 or to the server 6 through the mobile device 8 by the transceiver 37 of the storage box 3. The server 6 may provide the mobile device 8 with a message corresponding to the usage information, for example, a message about purchasing a replacement cartridge when the E-liquid is running out; alternatively, when the consumer is near an electronic cigarette store, marketing information about the cigarette which the consumer may need or want may be sent to the consumer through a location based service (LBS). Furthermore, according to the location and time of vaping, a marketing SMS including catering or service information may be sent to the consumer. For example, after obtaining information that many people are vaping electronic cigarettes at the same location, and the restaurant operator can send a marketing SMS about fast food or drink to the people's mobile devices 8 during customary meal hours.

The mobile device 8 may further include a user interface 82, and the user may input setting information through the user interface 82. For example, the user may input a maximum number of puffs of vaping the electronic cigarette per day, and according to the setting information, the mobile device 8 may send a control signal to the control chip 22 of the power supply module 2 through the transceiver 37 of the storage box 3, in order to stop the power source 21 from providing power to the atomizing component 13 when the number of puffs of vaping the electronic cigarette in the day reaches the maximum number. Therefore, the user may control personal usage habits by adjusting the setting information.

The mobile device 8 may also be used to identify the user by using application software. If the user is identified as a minor, the mobile device 8 sends a message to the control chip 22 to stop providing power to the atomizing component 13. Therefore, the purpose of preventing the person under a specific age from using the electronic atomizer can be achieved.

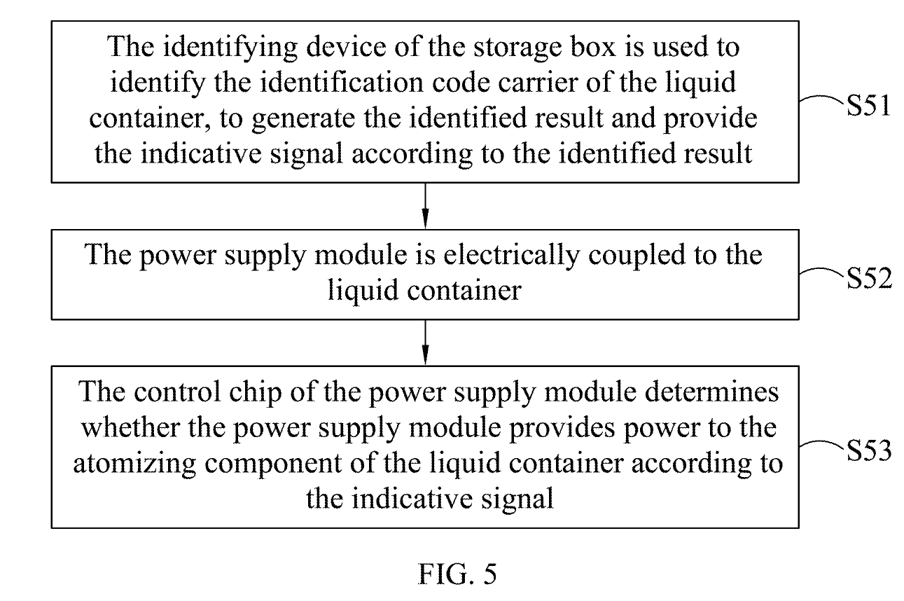

The following refers to FIG. 5, which is a flowchart showing the steps in an operation of the anti-counterfeiting method for the electronic atomizer of the present disclosure. As shown in FIG. 5, the anti-counterfeiting method includes the following steps.

In a step S51, the identifying device of the storage box is used to identify the identification code carrier of the liquid container, to generate the identified result and provide the indicative signal according to the identified result.

In a step S52, the power supply module is electrically coupled to the liquid container.

In a step S53, after the power supply module is electrically coupled to the liquid container, the control chip of the power supply module determines whether the power supply module provides power to the atomizing component of the liquid container according to the indicative signal.

The following refers back to FIG. 1. After the liquid container 1 has been placed into the receiving space of the storage box 3 through the opening, the identifying device 34 of the storage box 3 may identify the identification code carrier 15 of the liquid container 1 to generate the identified result. The identification manner may include the identifying device 34 using the public key to decrypt the identification code of the identification code carrier 15 encrypted by the private key. If the decryption fails, the identifying device 34 determines that the liquid container 1 is the counterfeit product and correspondingly generates the indicative signal S indicative of the counterfeit product. If the decryption succeeds, the identifying device 34 obtains the serial number of the liquid container 1 and correspondingly generates the indicative signal indicative of the genuine product.

After the power supply module 2 has been electrically coupled to the liquid container 1, the control chip 22 of the power supply module 2, according to the indicative signal S, determines whether the power source 21 provides power to the atomizing component 13 of the liquid container 1. That is, when the identified result indicates that the liquid container 1 is the genuine product, the control chip 22 enables the power source 21 to provide power to the atomizing component 13; however, when the identified result indicates that the liquid container 1 is the counterfeit product, the control chip 22 controls the power source 21 to not provide power to the atomizing component 13, thereby disabling atomization of the liquid 12 and preventing the user from using the counterfeit product.

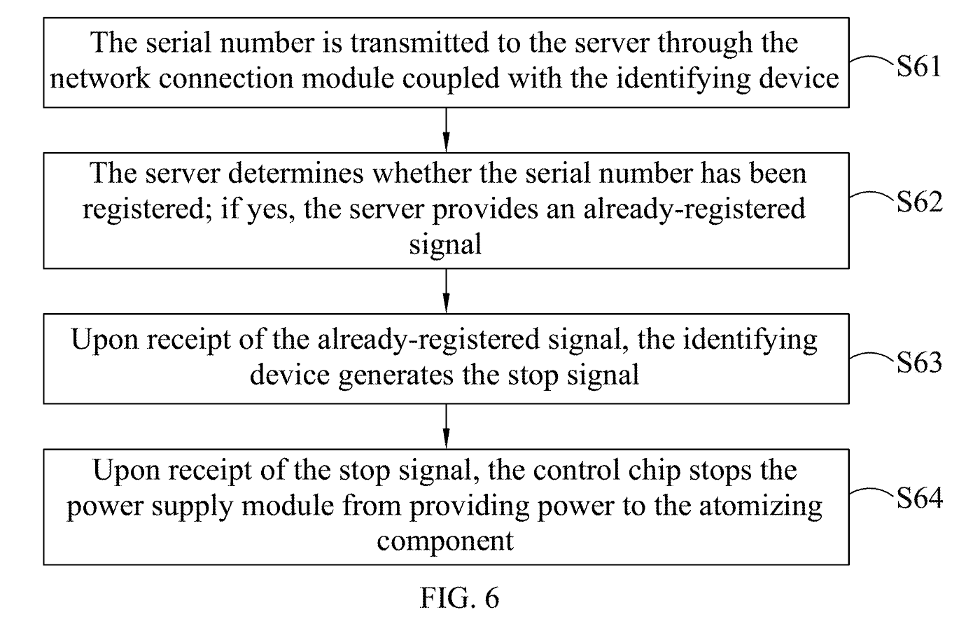

The following refers to FIG. 6, which is a second flowchart showing the steps in an operation of the anti-counterfeiting method for the electronic atomizer of the present disclosure. As shown in FIG. 6, the anti-counterfeiting method includes the following steps.

In a step S61, the serial number is transmitted to the server through the network connection module coupled with the identifying device.

In a step S62, the server determines whether the serial number has been registered; if yes, the server provides an already-registered signal.

In a step S63, upon receipt of the already-registered signal, the identifying device generates the stop signal.

In a step S64, upon receipt of the stop signal, the control chip stops the power supply module from providing power to the atomizing component.

The following description refers back to FIG. 4. The identifying device 34 transmits the indicative signal S including the serial number, to the server 6 through the network connection module 7. After receiving the serial number, the server 6 checks whether the serial number has been registered; that is, the server 6 determines that the liquid container 1 is the counterfeit product if the serial number of the identification code carrier 15 of the liquid container 1 has already been registered. If the serial number of the liquid container 1 has already been registered, the server 6 provides the already-registered signal to the identifying device 34 through the network connection module 7, and the identifying device 34 generates the stop signal to the control chip 22 of the power supply module 2, and the control chip 22 stops the power source 21 from providing power to the atomizing component 13, thereby disabling the atomization of the liquid 12 and preventing the user from using the counterfeit product.

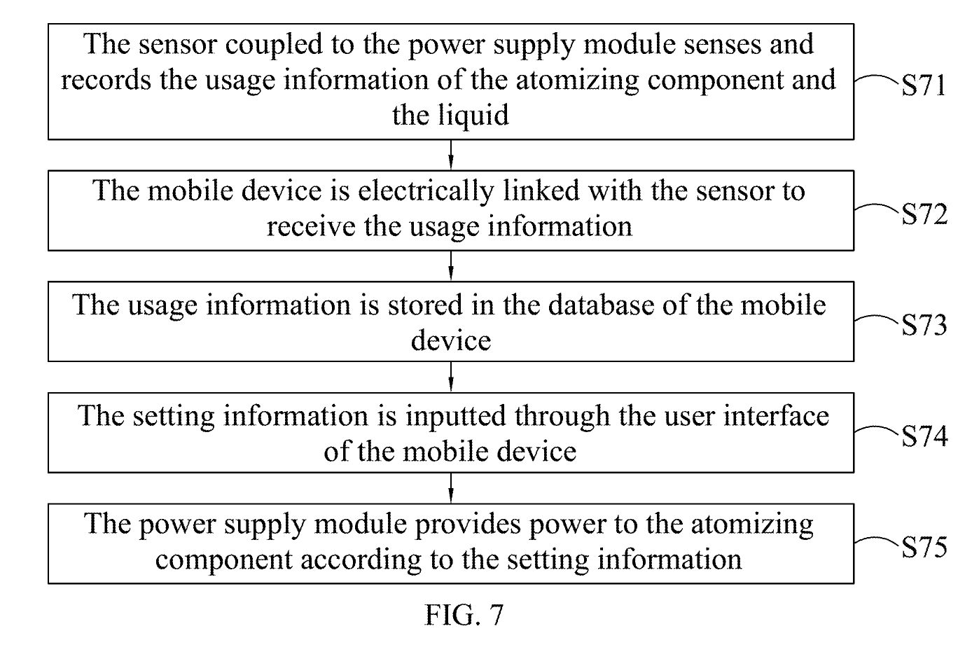

The following refers to FIG. 7, which is a third flowchart showing the steps in an operation of the anti-counterfeiting method for the electronic atomizer of the present disclosure. As shown in FIG. 7, the anti-counterfeiting method includes the following steps.

In a step S71, the sensor coupled to the power supply module senses and records the usage information of the atomizing component and the liquid.

In a step S72, the mobile device is electrically linked with the sensor to receive the usage information.

In a step S73, the usage information is stored in the database of the mobile device.

In a step S74, the setting information is inputted through the user interface of the mobile device.

In a step S75, the power supply module provides power to the atomizing component according to the setting information.

The following description refers back to FIG. 4. The sensor 24 which is electrically coupled to the power supply module 2 is used to sense and record the usage information of the atomizing component 13 and the liquid 12. The usage information includes information about the remaining amount of the liquid 12 calculated from the total number of sensed mouthfuls of using the electronic atomizer, a location where the electronic atomizer has been used, the user's usage habits including number of inhalations and duration of each usage session, as well as the times and frequency of the usage sessions. The usage information may be transmitted to the mobile device 8 through the network. When the power supply module 2 is placed into the receiving space of the storage box 3, the transceiver 37 of the storage box 3 transmits the usage information to the mobile device 8 through the network; or alternatively, the power supply module 2 has networking capacity to directly transmit the usage information to the mobile device 8 through WIFI or other wireless network.

After receiving the usage information, the mobile device 8 stores the usage information in the database 81 which may be provided by the application program of the mobile device 8.

The mobile device 8 includes the user interface 82, and the user may input the setting information through the user interface 82. For example, the user may input a maximum number of inhalations in the daily usage of the electronic atomizer, the times of the day that the electronic atomizer can be used, or set a daily reminder time for use of the electronic atomizer. The mobile device 8 provides the setting information to the control chip 22 of the power supply module 2 through the network, and the control chip 22 controls the power source 21 to provide power to the atomizing component 13 according to the setting information, or the mobile device 8 uses voice or vibration to remind user to use the electronic atomizer. Therefore, the user is able to define their own rules to fit their usage of the electronic atomizer.

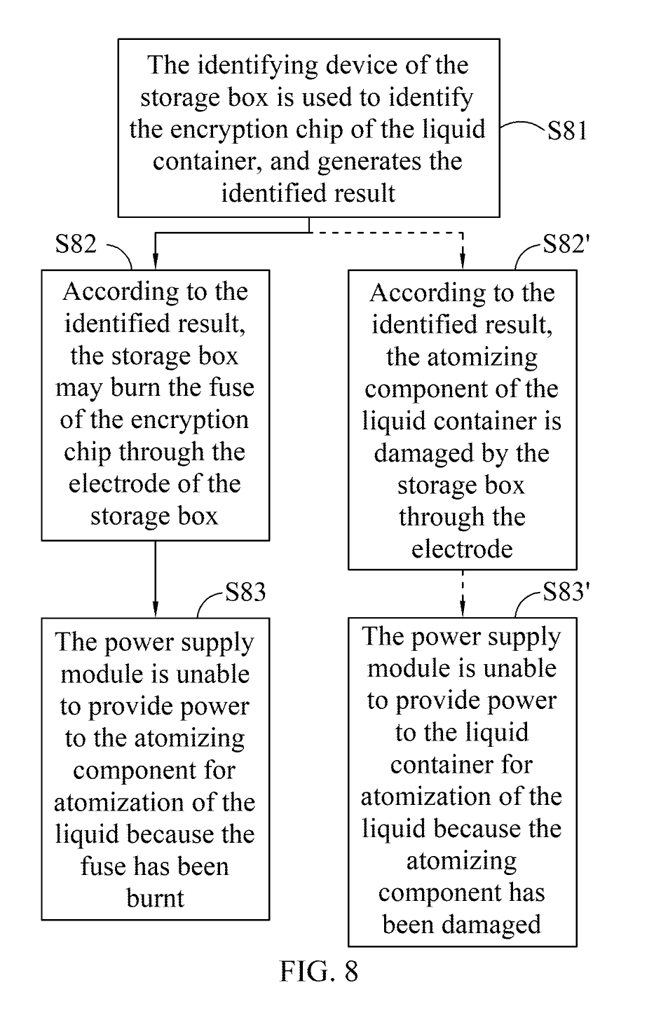

The following refers to FIG. 8, which is a fourth flowchart showing the steps in an operation of the anti-counterfeiting method for the electronic atomizer of the present disclosure. As shown in FIG. 8, the anti-counterfeiting method includes the following steps.

In a step S81, the identifying device of the storage box is used to identify the encryption chip of the liquid container, and generates the identified result.

In a step S82, according to the identified result, the storage box may burn the fuse of the encryption chip through the electrode of the storage box.

In a step S83, after the power supply module has been electrically coupled with the liquid container, the power supply module is unable to provide power to the atomizing component for atomization of the liquid because the fuse has been burnt.

The steps S82 and S83 may be replaced by steps S82′ and S83′.

In a step S82′, according to the identified result, the atomizing component of the liquid container is damaged by the storage box through the electrode.

In a step S83′, after the power supply module has been electrically coupled to the liquid container, the power supply module is unable to provide power to the liquid container for atomization of the liquid because the atomizing component has been damaged.

The following description refers back to FIG. 2. After the liquid container 1 has been placed into the receiving space of the storage box 3, the identifying device 34 of the storage box 3 identifies the encryption chip of the identification code carrier 15 of the liquid container 1. If the liquid container 1 is determined to be the counterfeit product according to the identified result, the fuse 16 of the encryption chip is burnt by the current flowing through the electrode 38 of the storage box 3. Therefore, after the power supply module 2 has been coupled to the liquid container 1 and the power supply module 2 has determined that the fuse 16 of the encryption chip of the liquid container 1 has been burnt, the power supply module 2 does not provide power to the atomizing component 13. The control chip 22 of the power supply module 2 can access the encryption chip to determine whether the fuse 16 of the encryption chip has been burnt; or alternatively, the storage box 3 can provide the indicative signal S to the control chip 22 to determine the same.

In an alternative embodiment, the atomizing component 13 of the liquid container 1 may be damaged according to the identified result, instead of burning the fuse 16 of the encryption chip, and thereby also achieving the purpose of preventing reuse of the liquid container 1.

In summary, the electronic atomizer, the anti-counterfeiting system and the anti-counterfeiting method of the present disclosure is able to determine whether the liquid container is the genuine product or the counterfeit product, so that the user can be protected from harm through use of the counterfeit product.

The present invention disclosed herein has been described by means of specific embodiments. However, numerous modifications, variations and enhancements can be made thereto by those skilled in the art without departing from the spirit and scope of the invention set forth in the claims.

What is claimed is:

1. An electronic atomizer, comprising:
a liquid container, configured to contain liquid to be atomized, and comprising an identification code carrier and an atomizing component;
a power supply module, configured to provide power to the atomizing component for atomization of the liquid; and
a storage box, comprising a receiving space and an identifying device configured to identify the identification code carrier of the liquid container which is placed in the receiving space.

2. The electronic atomizer of claim 1, wherein the power supply module comprises a control chip, and the identifying device generates an indicative signal after identifying the identification code carrier, and when the power supply module and the liquid container are electrically coupled with each other, the control chip determines whether the power supply module provides power to the atomizing component according to the indicative signal.

3. The electronic atomizer of claim 1, wherein the identifying device comprises a public key corresponding to a private key of the identification code carrier, and the identifying device is configured to decrypt the identification code carrier according to the public key, in order to identify a serial number stored in the identification code carrier.

4. The electronic atomizer of claim 2, wherein the identification code carrier is an encryption chip which comprises a fuse, and the storage box comprises an electrode configured to provide current to burn the fuse.

5. The electronic atomizer of claim 2, wherein the storage box comprises an electrode configured to provide current to damage the atomizing component.

6. An anti-counterfeiting system for an electronic atomizer, comprising:
the electronic atomizer of claim 1;
a network connection module, electrically coupled with the electronic atomizer and configured to transmit the indicative signal; and
a server, communicating with the network connection module through a network, and configured to receive the indicative signal comprising a serial number, and determine whether the serial number has been registered, and if the serial number has been registered, the server transmits an already-registered signal to the identifying device of the storage box, the identifying device generates a stop signal according to the already-registered signal, and upon receipt of the stop signal, the control chip stops the power supply module from providing power to the atomizing component.

7. An anti-counterfeiting method for an electronic atomizer, the electronic atomizer comprising a liquid container, a storage box and a power supply module, and the anti-counterfeiting method comprising the steps of:
identifying, by an identifying device of the storage box, an identification code carrier of the liquid container to generate an identification result, and providing an indicative signal according to the identification result;
electrically connecting the power supply module to the liquid container; and
determining, by a control chip of the power supply module, whether the power supply module should provide power to an atomizing component of the liquid container.

8. The anti-counterfeiting method of claim 7, wherein the step of identifying the identification code carrier, further comprises the steps of:
decrypting, by the identifying device, an identification code of the identification code carrier according to a public key;
when decryption fails, determining the liquid container with the identification code to be a counterfeit product and generating the indicative signal indicative of a counterfeit product; and
when decryption succeeds, obtaining a serial number of the liquid container and generating the indicative signal indicative of a genuine product.

9. The anti-counterfeiting method of claim 8, further comprising the steps of:
transmitting the serial number, through a network connection module connected to the identifying device, to a server;
determining, by the server, whether the serial number has been registered;
if the serial number has been registered, providing an already-registered signal by the server;
receiving the already-registered signal, and generating a stop signal by the identifying device; and
upon receipt of the stop signal, stopping the power supply module from providing power to the atomizing component, by the control chip.

10. A liquid container adapted to an electronic atomizer, comprising:
a liquid storage chamber, configured to accommodate fluid to be atomized;
an atomizing component, configured to atomize the fluid; and
an identification code carrier, comprising encrypted content;
wherein when decryption of the encrypted content fails, the atomizing component is burnt to disable the atomizing component and prevent atomization of the fluid.
